(19) United States
(12) Patent Application Publication
(10) Pub. No.: US 2008/0274200 A1
(43) Pub. Date: Nov. 6, 2008

(54) DOSE TITRATABLE LIQUID DOSAGE FORMS OF ACID LABILE DRUGS

(75) Inventor: Rajneesh TANEJA, Libertyville, IL (US)

Correspondence Address: DYKEMA GOSSETT PLLC, 39577 WOODWARD AVENUE, SUITE 300, BLOOMFIELD HILLS, MI, 48304-5086, US

(73) Assignee: TAP PHARMACEUTICAL PRODUCTS, Lake Forest, IL (US)

(21) Appl. No.: 12/048,570

(22) Filed: Mar. 14, 2008

Related U.S. Application Data
(63) Continuation of application No. 10/611,584, filed on Jul. 1, 2003, now abandoned.
(60) Provisional application No. 60/394,228, filed on Jul. 3, 2002.

(51) Int. Cl. A61K 9/16; A61K 31/4439; A61P 1/00

(52) U.S. Cl. 424/490; 514/338

(57) ABSTRACT

Provided herein are pharmaceutical formulations comprising micro-granules and a liquid suspension vehicle having a pH less than 6.0 and a viscosity sufficient to suspend the micro-granules. The micro-granules typically contain a PPI which therefore makes the above formulation, as well as kits for making the above formulation, useful for alleviating the symptoms of gastrointestinal disorders or diseases.

This application claims priority to U.S. Provisional Patent Application Ser. No. 60/394,228, filed Jul. 3, 2002, which is hereby incorporated by reference.

### TECHNICAL FIELD

The present invention relates to liquid dosage forms and in particular, relates to liquid dosage forms of proton pump inhibitors.

### BACKGROUND OF THE INVENTION

Many pharmaceutical compounds are susceptible to degradation in acidic environments. For example, antibiotics such as erythromycin; proton pump inhibitors (or “PPIs”) such as lansoprazole, or omeprazole; and pencreatin; are compounds that degrade in acidic environments and are therefore referred to as “acid labile”. Oral delivery of acid labile pharmaceutical compounds is challenging because the gastric pH is very acidic (typically between about pH 1.5 and 1.9). Under gastric conditions, acid-labile drugs typically degrade and are not readily available for uptake without being protected.

Due to the pH sensitivity of acid labile drugs, they typically are administered in a form that protects the drug from the acidic gastric environment. Enteric coatings are probably the most widely used method of protecting acid-labile drugs from gastric degradation. Enteric coating methods typically form a barrier around drug particles, or an entire dosage form containing an acid-labile drug, with a coating that does not dissolve upon introduction to the low pH of the gastric environment. Such enteric coatings typically dissolve at a pH greater than 6, such as that found in the upper small intestine where the acid labile drugs are released in an environment where they will not significantly degrade, and therefore can be absorbed. Drugs requiring enteric coatings are often times formulated as capsules or tablets that are difficult to administer to patients who have difficulty swallowing such as pediatric patients, or patients who cannot swallow at all such as critically ill patients.

Moreover circumstances exist that warrant a partial dose of an acid-labile drug such as, for example, a PPI. For example, patients suffering from hepatic insufficiency are often times recommended to take one-half of the normal dose of a PPI. While solid dosage forms of PPI's are available, these dosage forms are not readily amenable to accurate and consistent fractioning. In particular, there is no means for readily providing an accurate portion of the active ingredient contained in the dosage forms that contain PPI's.

Notwithstanding the above, attempts have been made for formulate liquid dosage forms of acid-labile drugs. For example, U.S. Pat. No. 5,840,737 recommends dissolving the contents of commercially available capsules containing enterically coated pellets of a PPI in a solution of sodium bicarbonate buffer. In practice, the above method requires a practitioner to open a capsule and release the enterically coated PPI into the buffer. After the contents of the capsule and buffer are combined, the mixture is swirled or mixed for between approximately 20 to 30 minutes so that the enteric coating around the PPI dissolves due to the buffer's relatively high pH. Once the enteric coating is dissolved, the PPI is relatively stabile in the buffer and is able to be administered to a patient. However, the volume of buffer used in this practice is relatively large and can produce stomach gases and therefore belching which is detrimental to individuals suffering from gastro-esophageal reflux disease (GERD), one of the disease states a PPI is intended to alleviate. Additionally, the buffer employed typically is a separate component that adds to the cost of providing such a formulation. Moreover, when given orally, the taste of such a solution is unpleasant. Also, great care must be taken to completely dissolve the enteric coating layer from the enterically coated PPI since undissolved components of an enteric coating layer tend to form sticky globules that can stick together.

Unfortunately, none of the presently available liquid dosage forms of PPI's have the ability to accurately dispense portions of the active ingredient.

There is therefore a need for a liquid dosage form of a PPI that maintains the efficacy of the active component, is easily administered to patients who have difficulty swallowing solid dosage forms, and can be dispensed such that a portion of the active ingredient can accurately be dosed from a bulk liquid.

### SUMMARY OF THE INVENTION

The present invention provides PPIs in a liquid dosage form that are dose titratable and easily administered to patients having difficulty swallowing who are suffering from gastrointestinal disorders. The formulations generally comprise micro-granules that comprise an acid-labile drug coated with an enteric coating; and a liquid suspension vehicle having a pH less than 6.0 and having a viscosity sufficient to suspend the micro-granules. The formulation can be provided in a kit containing separate containers of the various components of the formulation.

### DETAILED DESCRIPTION OF THE INVENTION

The present invention provides liquid formulations of PPIs. Partial doses of the active ingredient can accurately and reliably dispensed from the bulk formulations (sometimes referred to as dose titrating). Hence, patients that are compromised in their ability to swallow can be given partial doses of an active ingredient that generally is easy to swallow.

Generally, the formulation comprises micro-granules of an enterically coated PPI and a liquid suspension vehicle having a pH that will not dissolve the enteric coating and has a viscosity that is capable of uniformly suspending the micro-granules for a sufficient time to dose titrate the bulk suspension of micro-granules. The micro-granules typically will comprise a PPI protected by an enteric coat. Any of the well known PPIs are suitable for use in the present invention and include, but are not limited to, lansoprazole, omeprazole, and pantoprazole. Combinations of PPIs as well as enantiomers and prodrugs of PPIs are also suitable for use in the present formulations. Such drugs may be formulated with other active or inactive ingredients before being enterically coated. For example, stabilizers such as salts of group I or group II metals such as, for example, magnesium oxide, magnesium hydroxide, calcium carbonate, or sodium bicarbonate may be used in such formulations to maintain the integrity of the active drug; fillers such as talc; as well as sugars and other excipients such as sucrose, mannitol, and microcrystalline cellulose, may also be part of such formulations.

Additionally, multiple coatings may be applied to the core formulation prior to applying the enteric coating layer. Additional coatings typically are employed to protect the acid labile drug in cases where it may react with the enteric coating material. Hence, the additional coat is between the acid labile drug core and the enteric coat. Materials that typically are employed for this purpose include, but are not limited to hydroxy propyl cellulose and hydroxy propyl methyl cellulose. The use of additional coating layers in a micro-granule is largely a matter of choice for those skilled in the art based upon the composition of the acid labile drug and enteric coating material employed. Such determinations are routinely made empirically by performing side-by-side stability studies on enterically coated micro-granules having sub-coatings and similarly composed micro-granules without sub-coatings.

Enteric coatings and methods for applying enteric coats are well known in the art. Enteric coatings generally comprise ingredients that do not dissolve in environments having a pH less than 6.0. Typically, enteric coatings of the present invention will protect the acid-labile drug in the pH of the liquid composition as well as the gastric environment. Any of the well known enteric coating materials are suitable for use with the present invention and may include, for example, waxes such as stearic acid, palmattic acid, and behenic acid; and polymers such as cellulose acephthalate, polyvinyl acetate phthalate, hydroxypropyl methyl cellulose acetate.

Microgranules may take many different forms depending upon the granulation and sieving procedures employed but typically are, for the most part, spherical in nature and have a size range of between 100 μm and 900 μm, more preferably between 100 μm and 700 μm, and most preferably between 200 μm and 500 μm.

Liquid suspension vehicles that may be employed according the present invention have a pH that will not dissolve the enteric coating of the microgranules. Typically a liquid suspension vehicle having a pH of less than 6.0 will not dissolve an enteric coating. Liquid suspension vehicles may inherently have a pH of less than 6.0 or additional ingredients such as acidic excipients may be added to a liquid vehicle to achieve a pH of less than 6.0. Appropriately pHed buffers are well suited acidic excipients that can be employed for this purpose and any of the well known buffers are suitable for maintaining the pH of liquid below 6.0. Typically, the pH of the liquid suspension vehicle is less than 6.0, preferably less than 5.5, and more preferably less than 5.0.

The liquid suspension vehicle also serves to maintain the homogeneity of the micro-granules in the formulation. In large part, this ability is due to the viscosity of the liquid vehicle. Typically, the viscosity of the liquid suspension vehicle is such that after a brief mixing of the micro-granules in the vehicle, the micro-granules will be homogenously dispersed in the liquid vehicle for a sufficient time to dispense a uniform dose of the microgranules from the formulation. A so-called “brief mixing” would include swirling, shaking, or otherwise agitating the contents of the formulation for at least 30 seconds, more preferably at least 60 seconds, and more preferably at least 90 seconds. A dose of the resulting suspension of micro-granules typically may be dispensed shortly after mixing. Hence, the viscosity of the liquid vehicle can maintain the homogeneity of the micro-granules in the liquid vehicle for at least 15 seconds, preferably at least 30 seconds, more preferably one minute, and most preferably 2 minutes. The phrase “homogeneously dispersed” or similar language used to describe the micro-granules in the liquid suspension vehicle means that at least 90% of the intended concentration of the active drug, and more preferably at least 95% of the intended concentration of the active drug is present throughout the formulation. Thus, for example, if the concentration of a PPI in a particular formulation were 100 mg/ml, a sampling of a milliliter of the bulk formulation at any particular site in the container should contain at least 90 mg of the PPI. Formulations having viscosities of greater than 500 cP, more preferably greater than 800 cP, and more preferably greater than 900 cP, are suitable for the above purposes. These viscosities are based upon a Brookfield type viscometer equipped with a #3 spindle rotating at 5 rpm.

The liquid suspension vehicle may inherently have a sufficient viscosity to achieve the above purpose. However, other agents may be added to a liquid to achieve a desirable viscosity. Various thickening agents for increasing the viscosity of a liquid formulation are well known. For example, cellulose and its derivatives; gums such as guar and xanthan; alginates; and polymers such as polyvinyl alcohol, polyvinyl pyrollidine, and poloxamer are well known excipients that can be employed to increase the viscosity of a liquid. Such thickening agents may be added to a liquid until the desired viscosity is achieved.

Due to the nature of the present formulation the volume of the liquid suspension vehicle employed is extremely variable. While there is no upper limit to the volume of the liquid vehicle employed, practical considerations typically suggest volumes of less than 500 ml, preferably less than 100 ml, more preferably less than 50 ml and most preferably less than 15 ml.

The concentration of the micro-granules in a formulation is also variable and largely dependent upon the size of the micro-granule and the therapeutically effective amount of the PPI in the micro-granule. Therapeutically effective amounts of PPI's, for example, can vary between 5 mg and 300 mg depending upon the PPI employed. Generally, however, “therapeutically effective amount(s)” means an amount of a drug given to a patient at a frequency that alleviates the gastrointestinal symptoms experienced by the patient receiving such therapy. The specific therapeutically effective amount for any particular patient will depend upon a variety of factors including the disorder being treated; the severity of the disorder; the activity of the specific compound employed; the specific composition employed; the age, body weight, general health, sex and diet of the patient; the time of administration; route of administration; the rate of excretion of the specific compound employed; the duration of the treatment; the drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved. These parameters can then be employed to appropriately dose a particular patient such that a patient receives the desired effect.

Many configurations for the formulations are possible. For example, the micro-granules can be formulated into a fast dissolving tablet such as those found in U.S. Pat. Nos. 5,464,632 and 6,299,904 which are herein incorporated by reference. Such tablets may also contain an acidic excipient such that when the tablet is placed in water the micro-granules and acidic excipient are released. The tablets may also be formulated with thickening agents as well. As a result, upon dissolution of the tablets in water, for example, the micro-granules will be in a liquid suspension vehicle having an appropriate viscosity and pH. Alternatively, sachet formulations are also suitable for producing a formulation comprising micro-granules and a liquid vehicle having a pH of less than 6.0, as well as a sufficient viscosity. Sachets typically are packaged dry ingredients that, for present purposes, could contain enterically coated micro-granules of a PPI, an acidic excipient, and a thickening agent. Similarly to a fast dissolving tablet, such a sachet formulation simply could be placed in water to create a formulation comprising micro-granules and a liquid vehicle having a pH of less than 6.0, as well as an appropriate viscosity. As a further alternative the liquid may already have the appropriate pH and viscosity and the microgranules could be added to such a liquid to produce the formulation. Of course, other ingredients such as flavoring agents, sweeteners, preservatives, coloring agents, and the like could be added to such formulations.

The compositions of the present invention may be provided as kits containing separately packaged containers of the components of the composition. For example, a kit may contain a vial of liquid and a tablet containing dry ingredients of the formulation such as the enterically coated micro-granules, a thickening agent and an acidic excipient. Upon dissolving the tablet in the liquid a complete liquid formulation is formed. It will be understood of course that the formulation may come as separate components, but the separate components should be mixed to form a liquid formulation comprising the microgranules in a liquid vehicle having a pH and viscosity such as those specified above prior to administration. In cases where kits are provided, the kits may come with instructions for appropriate mixing of the dry and liquid components and administration utensils such as, for example, a syringe. Additionally, when kits are provided, it is preferable that the dry ingredients completely disperse in the liquid ingredients in less than 5 minutes, preferably in less than 2 minutes and most preferably in less than 1 minute.

Preferably, compositions of the present invention comprising a PPI are provided to patients experiencing gastrointestinal disorders such as acid reflux disease, gastro-esophogeal reflux disease, peptic and duodenal ulcers, or any other gastrointestinal disorder for which PPI's are indicated to thereby alleviate such disorders. Hence, methods are provided for treating gastrointestinal disorders comprising administering a composition of the present invention comprising a PPI to a patient in need of such a therapy such as those experiencing gastrointestinal disorders.

The following examples are provided to further illustrate the present invention and not intended to limit the invention.

### EXAMPLES

### Example 1

### Suspension of Microgranules in Various Liquid Suspension Vehicles

In this example, micro-granules containing Lansoprazole® (a PPI) were suspended in various liquid suspension vehicles to determine if a uniform dose could be dispensed from the bulk suspension.

In particular, Lansoprazole Fast Dissolving Tablets (TAP Pharmaceutical Products Inc., Lake Forest Ill.) were dissolved in various volumes of water with a variety of thickening agents. Lansoprazole Fast Dissolving Tablets (LFDT) comprise enterically coated microgranules of Lansoprazole, mannitol, sweetener, and acidifier. Due to the acidic excipients in LFDT, upon dissolution the pH of the resulting solution is less than 6.0. Typically, dissolution of an LFDT tablet in 30 ml of water results in a pH of less than 5.0. The thickening agents used in this experiment were Smuckers strawberry syrup, Citrucel (commercially available), and the inactive ingredients from Lansoprazole Sachet (TAP). Lansoprazole sachet inactive ingredients contain approximately 2.0 gm xanthan gum as a thickening agent.

Several suspensions were made using the various components specified above and LFDT tablets containing 30 mg of Lansoprazole. The suspensions were made by placing distilled water and one LFDT tablet into a 25 ml bottle and mixing until the tablet was disintegrated. The thickening agent was then added to the 25 ml bottle containing the suspended LFDT tablet and vigorously mixed. Samples of the suspensions were then taken and prepared for HPLC analysis to determine the concentration of Lansoprazole in the sample. This data was compared to the concentration that theoretically should have been in the sample (i.e. 30 mg/volume of the suspension) to arrive at the “% of theory” presented in the tables below. Samples were analyzed at least in duplicate, and most often triplicate, and the values reported are the average of the % of theory measurements. Relative standard deviations (RSD) for the reported % of theory values are also provided. The actual amounts of ingredients tested according to the above procedure and the average % of theory are found in Table 2, below.

TABLE 2

| | Thickener | Water | | |
|---|---|---|---|---|
| Thickener | Amount (gm) | Volume (ml) | % of Theory | RSD |
| Citrucel | 4 | 10 | 111.7 | 2.8 |
| Citrucel | 4 | 10 | 108.9 | 5.4 |
| Citrucel | 3 | 10 | 116.3 | 6.1 |
| Citrucel | 3 | 10 | 103.6 | 8.5 |
| Citrucel | 2 | 10 | 97.3 | 8 |
| Strawberry | 10 | 5 | 106.7 | 2.8 |
| Syrup | | | | |
| Strawberry | 10 | 5 | 99.5 | 2.1 |
| Syrup | | | | |
| Strawberry | 10 | 3 | 94.6 | 3.7 |
| Syrup | | | | |
| Strawberry | 10 | 2 | 106.4 | 5.0 |
| Syrup | | | | |
| Strawberry | 10 | 5 | 110.0 | 4.5 |
| Syrup | | | | |
| Stawberry | 10 | 5 | 109.1 | 8.3 |
| Syrup | | | | |
| Strawberry | 15 | 1 | 102.7 | 1.9 |
| Syrup | | | | |
| Sachet Inactive | 0.5 | 10 | 106.7 | 4.0 |
| Ingredients | | | | |
| Sachet Inactive | 1 | 10 | 95.9 | 6.8 |
| Ingredients | | | | |
| Sachet Inactive | 1 | 10 | 103.3 | 1.9 |
| Ingredients | | | | |
| Sachet Inactive | 2 | 10 | 95.2 | 13.6 |
| Ingredients | | | | |
| Sachet Inactive | 3 | 10 | 98.4 | 8.1 |
| Ingredients | | | | |

As shown by the data in Table 2, the concentration of Lansoprazole in a given sample from any of the suspensions was greater than 90% of the theoretical amount of Lansoprazole that should have been in the sample.

### Example 2

### Reproducibility of Drug Amount in Suspension

In this example, ten suspensions were made and a single sample of each suspension was analyzed in the same manner as in Example 1. Each sample was prepared with 10 ml of distilled water, 1 LFDT tablet, and 2 gm of sachet inactive ingredients. The % of theory of each of the ten suspensions in shown below in Table 3. Additionally, two additional samples were taken from sample number 10 to determine if the % of theory in multiple aliquots from a single suspension were reproducible. These results are shown in Table 4, below.

TABLE 3

| Suspension | % of Theory |
|---|---|
| 1 | 104.7 |
| 2 | 107.3 |
| 3 | 107.0 |
| 4 | 104.5 |
| 5 | 104.9 |
| 6 | 105.4 |
| 7 | 101.4 |
| 8 | 101.2 |
| 9 | 104.2 |
| 10 | 104.4 |

TABLE 4

| Additional Sample | % |
|---|---|
| from Suspension 10 | of Theory |
| 1 | 98.9 |
| 2 | 100.7 |

The average % of theory for the 10 suspensions reported above was 104.5 (RSD 1.90). The average % of theory for the multiple samples from suspension number 10 was 101.3 (RSD 2.8). Hence, samples from multiple distinct suspensions yielded a consistently similar % of theory. Additionally, the % of theory from multiple samples from a single suspension was also consistent.

### Example 3

### Acid Resistance of Microgranules in Liquid Suspension Vehicles

In this example, the stability of the active component in the formulation was evaluated. Specifically, the stability of the acid-labile drug Lansoprazole in the formulation was determined.

One LFDT tablet was dispersed in 10 ml of distilled water before 2 gm of sachet inactive ingredients were added to the dispersed LFDT tablet. The suspension was mixed for one minute. The suspension was poured into a dissolution vessel containing 500 ml of 0.1 N HCl that was pre-warmed to 37±0.5° C. Prior to placing the LFDT suspension in the acid solution, 10 ml of the 0.1 N HCl was removed and used to rinse the vessel containing the LFDT suspension twice (5 ml per wash). After addition of the rinsing solutions, a paddle in the dissolution vessel was set at 75 rpm and allowed to mix for 60 minutes. After mixing, a 10 ml aliquot was taken from the dissolution vessel and filtered through a 0.45 um filter. The filtrate was tested spectrophotometrically for absorbance at 306 nm (the wavelength at which by-products of Lansoprazole degradation show absorbance) and compared to the absorbance spectra for LFDT dissolved in distilled water. Table 5 shows the % released Lansoprazole for 4 replicates of the formulation including the sachet inactive ingredients and a two replicates of LFDT tablets suspended in distilled water alone treated in the same manner as the LFDT tablets in water and sachet inactive ingredients.

TABLE 5

| Sample # | Identification | % released |
|---|---|---|
| 1 | LFDT alone | 6 |
| 2 | LFDT alone | 5 |
| 3 | LFDT & Sachet Inactives | 7 |
| 4 | LFDT & Sachet Inactives | 5 |
| 5 | LFDT & Sachet Inactives | 6 |
| 6 | LFDT & Sachet Inactives | 5 |

As shown in Table 5, the sachet inactive ingredients did not show any significant impact on the release of Lansoprazole from the enterically coated microgranules.

### Example 4

### Formulation Viscosity

In this example, various amounts of a thickening agent (sachet inactive ingredients) were added to distilled water and the viscosities of the resulting solutions measured. The viscosity was measured using a Brookfield viscometer equipped with a standard #3 spindle and temperature probe. 150 ml of each individual sample was placed in a 200 ml tall-form beakers and the viscosities measured with the spindle rotating at 5 rpm.

The ratio of ingredients in each 150 ml sample (grams of sachet inactive ingredients-to-milliliters of water), time for reading, temperature, % scale, and viscosity measurements are provided in Table 6 below.

TABLE 6

| Ratio of | Reading Time | Temp | | Viscosity |
|---|---|---|---|---|
| Ingredients | (minutes) | (° C.) | % Scale | (cP) |
| 0.75 gm:10 ml | 8 | 19.0 | 4 | 959.8 |
| 1.00 gm:10 ml | 8 | 19.0 | 6.9 | 1632 |
| 1.5 gm:10 ml | 8 | 18.8 | 15.1 | 3551 |
| 2.0 gm:10 ml | 8 | 18.6 | 24.1 | 5759 |
| 3.0 gm:10 ml | 8 | 18.4 | 35.8 | 8614 |

Analyzing the viscosity results presented above with the results of Example 1 demonstrates that viscosities of greater than 959 (using the viscometer parameters cited above) are sufficient to produce a formulation that is dose titratable. Specifically, samples taken from the formulation produced in example 1 using 0.5 gm of sachet inactive ingredients to 10 ml of water contained greater than 90% of the theoretical concentration of Lansoprazole. Additionally, the viscosity results presented in Table 6 show that a formulation made with a greater amount of thickener (i.e. 0.75 gm:10 ml) had a viscosity of 959. Hence, viscosities above 900 have sufficient ability to maintain micro-granules in suspension for sufficient time to dispense a dose of the suspension that has at least 90% of the expected active ingredient. Extrapolating from the above suggests that viscosities (using the viscometer parameters above) of greater than 500 should be sufficient to suspend micro-granules sufficiently to dose titrate the bulk solution.

While the invention has been described in detail and with reference to specific embodiments, it will be apparent to one skilled in the art that various changes and modifications may be made to such embodiments without departing from the spirit and scope of the invention.

What is claimed is:

1. A composition comprising:
(a) micro-granules comprising a proton pump inhibitor coated with an enteric coating; and
(b) a liquid suspension vehicle having a pH less than 6.0 and having a viscosity sufficient to suspend the micro-granules.

2. The composition of claim 1 wherein the micro-granules are between 100 μm and 900 μm.

3. The composition of claim 1 wherein the composition has a viscosity of greater than 500 cP.

4. The composition of claim 3 wherein the active ingredient is lansoprazole.

6. A method of treating a patient with a gastrointestinal disorder comprising providing to the patient a composition comprising micro-granules of an enterically coated proton pump inhibitor and a liquid suspension vehicle having a pH less than 6.0 and a viscosity sufficient to suspend the micro-granules.

7. A kit comprising:
a) a first container comprising micro-granules comprising a proton pump inhibitor coated with an enteric coating; and
b) a second container comprising a liquid;
wherein the first or the second container further comprises an acidic excipient and wherein the first or second container further comprises a thickening agent such that when the contents of the first and second container are combined, the resulting liquid has a viscosity sufficient to suspend the micro-granules.

8. The kit of claim 7 wherein the proton pump inhibitor is lansoprazole.

9. The kit of claim 8 wherein the viscosity of the liquid is greater than 500 cP.

10. The kit of claim 7 wherein the micro-granules are between 100 μm and 900 μm.
